(19) United States
(12) Patent Application Publication
(10) Pub. No.: US 2016/0339154 A1
(43) Pub. Date: Nov. 24, 2016

(54) GRAPHENE-BASED MOLECULAR/ENZYMATIC INTEGRATED CATALYSTS

(71) Applicants: THE REGENTS OF THE UNIVERSITY OF CALIFORNIA, Oakland, CA (US); THE REGENTS OF THE UNIVERSITY OF MICHIGAN, Ann Arbor, MI (US)

(72) Inventors: Xiangfeng Duan, Los Angeles, CA (US); Yu Huang, Los Angeles, CA (US); Teng Xue, Los Angeles, CA (US); Mark E. Meyerhoff, Ann Arbor, MI (US)

(73) Assignees: The Regents of the University of California, Oakland, CA (US); The Regents of the University of Michigan, Ann Arbor, MI (US)

(21) Appl. No.: 15/113,940

(22) PCT Filed: Jan. 27, 2015
(86) PCT No.: PCT/US2015/013118

§ 371 Date: Jul. 25, 2016

Related U.S. Application Data
(60) Provisional application No. 61/932,091, filed on Jan. 27, 2014.

(51) Int. Cl. A61L 33/02; B01J 31/18; C12N 11/14; A61L 27/54; A61L 31/08; A61L 31/16; A61L 27/30; B01J 31/00; A61L 33/00

(52) U.S. Cl. CPC A61L 33/025; B01J 31/003; B01J 31/183; C12N 11/14; A61L 33/0041; A61L 33/0047; C12Y 101/03004; A61L 33/0082; A61L 31/084; A61L 31/16; A61L 27/303; A61L 27/54

(57) ABSTRACT

Described here is a graphene-catalysts conjugate, comprising: a graphene support; a first catalyst conjugated to the graphene support; and a second catalyst conjugated to the graphene support, and the second catalyst is different from the first catalyst. In some embodiments, the first catalyst and the second catalyst correspond to a tandem catalytic system to drive a chemical transformation.

### CROSS-REFERENCE TO RELATED APPLICATION

This application claims the benefit of U.S. Provisional Application No. 61/932,091, filed Jan. 27, 2014, the disclosure of which is incorporated herein by reference in its entirety.

### STATEMENT REGARDING FEDERALLY SPONSORED RESEARCH OR DEVELOPMENT

This invention was made with Government support under EB000783, OD004342, OD007279, awarded by the National Institutes of Health. The Government has certain rights in the invention.

### BACKGROUND

Biological systems can often drive complex chemical transformations under mild conditions (e.g., aqueous solution, physiological pH, room temperature and atmospheric pressure), which is difficult to achieve in conventional chemical reactions. This ability is generally empowered by a series of synergistic protein catalysts that can facilitate reaction cascades through complex metabolic pathways. There is significant interest in exploring molecular assemblies and conjugated catalytic systems as analogs to functional proteins that can facilitate chemical transformation under biologically mild conditions. Although “artificial enzymes” have been studied for decades, catalysts mimicking true enzymes for designated and complex reaction pathways have been much less frequently explored. The integration of enzymatic catalysts with molecular catalysts could create functional tandem catalytic systems for important chemical transformations not otherwise readily possible. Despite the significant interest, it is quite challenging to build a system that can allow enzymatic catalysts and molecular catalysts to operate synergistically under the same conditions (e.g., aqueous solutions and physiological pH).

It is against this background that a need arose to develop the graphene-based catalysts described herein.

### SUMMARY

The integration of multiple synergistic catalytic systems can allow the creation of biocompatible enzymatic mimics for cascading reactions under physiologically relevant conditions. In some embodiments, this disclosure is directed to the design of a graphene-hemin-glucose oxidase (GOx) conjugate as a tandem catalyst, in which graphene functions as a support to integrate molecular catalyst hemin and enzymatic catalyst GOx with retained functionality for biomimetic generation of antithrombotic species. The monomeric hemin can be conjugated with graphene through π-π interactions to function as an effective catalyst for the oxidation of endogenous L-arginine by H₂O₂. Furthermore, GOx can be covalently linked onto graphene for local generation of H₂O₂ through the oxidation of blood glucose. Thus, the integrated graphene-hemin-GOx catalysts can readily allow the continuous generation of nitroxyl, an antithrombotic species, from physiologically abundant glucose and L-arginine. Lastly, the conjugates can be embedded within polyurethane to provide a long-lasting antithrombotic coating for blood contacting biomedical devices.

Another aspect of some embodiments of the disclosure relates to a graphene-based tandem catalyst, which comprises at least two different molecular and/or enzymatic catalysts conjugated to a graphene support covalently or non-covalently, wherein a first catalyst catalyzes a first reaction and a second catalyst catalyzes a second reaction different from the first reaction, and wherein at least one product of the first reaction participates in the second reaction. In some embodiments, the first catalyst is a molecular catalyst, while the second catalyst is an enzymatic catalyst, or vice versa. In some embodiments, the first catalyst is conjugated to the graphene surface, for example, by π-π interactions, while the second catalyst is conjugated to the edges and/or defect sites of graphene, for example, by covalent bonds, or vice versa. In some embodiments, the graphene-based tandem catalyst is adapted to modify the physiological level of at least one compound with antithrombotic property. In some embodiments, the graphene-based tandem catalyst is adapted to modify the physiological level of at least one compound without antithrombotic property.

Another aspect of some embodiments of the disclosure relates to a biocompatible film comprising the graphene-catalysts conjugate described herein. The biocompatible film can comprise, for example, a polymer layer embedded with the graphene-catalysts conjugate. The biocompatible film can comprise, for example, a polymer layer coated with the graphene-catalysts conjugate. The polymer can comprises, for example, one or more biocompatible polymers known in the art. In one embodiment, the polymer comprises polyurethane.

Another aspect of some embodiments of the disclosure relates to a biomedical device comprising the graphene-catalysts conjugate described herein. The biomedical device can comprise, for example, a surface coating comprising the graphene-catalysts conjugate. The biomedical device can comprise, for example, a porous substrate embedded with the graphene-catalysts conjugate. The biomedical device can comprise, for example, a reservoir storing the graphene-catalysts conjugate. The biomedical device can be, for example, an implant, a catheter, a vascular graft, a biosensor, or a heart valve.

Another aspect of some embodiments of the disclosure relates to a method for making the graphene-catalysts conjugate described herein, comprising providing a conjugate comprising a graphene support conjugated to a first catalyst, and covalently linking a second catalyst to the graphene support. In some embodiments, the first catalyst is hemin which is conjugated to the graphene support through π-π interactions. In some embodiments, the second catalyst is glucose oxidase, wherein the glucose oxidase is covalently linked to the graphene support through a coupling agent. In one embodiment, the coupling agent is N-Hydroxysuccinimide and 1-Ethyl-3-(3-dimethylaminopropyl)carbodiimide (NHS-EDC).

Another aspect of some embodiments of the disclosure relates to a method for making the biocompatible film described herein, comprising coating a surface with a composition comprising the graphene-catalysts conjugate. Coating methods known in the art can be used, such as spin coating. In some embodiments, the composition comprises at least one biocompatible polymer mixed with the graphene-catalysts conjugate.

Another aspect of some embodiments of the disclosure relates to a method for making the biomedical device described herein, comprising coating at least one surface of the biomedical device with a composition comprising the graphene-catalysts conjugate. In some embodiments, the composition comprises at least one biocompatible polymer mixed with the graphene-catalysts conjugate.

Another aspect of some embodiments of the disclosure relates to a method for making the biomedical device described herein, comprising embedding a composition comprising the graphene-catalysts conjugate into a porous substrate, wherein the porous substrate embedded with the graphene-catalysts conjugate forms part of the biomedical device.

Another aspect of some embodiments of the disclosure relates to a method for making the biomedical device described herein, comprising disposing a composition comprising the graphene-catalysts conjugate in a reservoir of the biomedical device, wherein in physiological condition the reservoir is in fluidic communication with the environment outside the biomedical device.

Another aspect of some embodiments of the disclosure relates to a method for improving the antithrombotic property of an implant, comprising coating the implant with a composition comprising the graphene-catalysts conjugate. In some embodiments, the method comprises coating a catheter, a vascular graft, a biosensor, or a heart valve with the composition comprising the graphene-catalysts conjugate, thereby improving the antithrombotic property thereof.

Another aspect of some embodiments of the disclosure relates to a method for producing at least one antithrombotic compound in vivo, comprising administering a composition comprising the graphene-catalysts conjugate described herein into a human patient, wherein the graphene-catalysts conjugate catalyzes the production of at least one antithrombotic compound, such as nitroxyl. In some embodiments, the method comprises implanting into the human patient an implant which is coated or embedded with the composition comprising the graphene-catalysts conjugate.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

FIG. 1 is a schematic illustration of graphene-hemin-GOx conjugates. Monomeric hemin molecules are conjugated with graphene through π-π interactions to function as an effective catalyst for the oxidation of L-arginine; and GOx is covalently linked to graphene for oxidation of glucose and local generation of H₂O₂.

FIG. 2 shows graphene-hemin conjugate catalyzed oxidation of L-arginine. a. A schematic illustration of graphene-hemin catalyzed L-arginine oxidation to produce nitroxyl. b. Relative fluorescence spectra at different reaction time obtained by DAF Assay. c. The nitroxyl concentration was measured using a DAF assay. The “A” line represents product formation using graphene-hemin conjugate catalyst. The “B” line represents product formation using free hemin catalyst. The “C” line represents product formation in a control experiment without any catalyst.

FIG. 3 shows nitroxyl generation behavior of graphene-hemin-GOx conjugates. a. Nitroxyl generation of graphene-hemin-GOx and control experiments. The production of nitroxyl was quantified using a DAF assay. The “A” line, graphene-hemin-GOx in glucose and L-arginine; “B” line, graphene-hemin in glucose and L-arginine; “C” line, graphene-GOx in glucose and L-arginine; “D” line, graphene-hemin-GOx in glucose; “E” line, graphene-hemin-GOx in L-arginine. b. Real time nitroxyl production catalyzed by graphene-hemin-GOx and the recyclability of the graphene-hemin-GOx catalysts.

FIG. 4 shows antithrombotic behavior of biocompatible films containing graphene-hemin-GOx conjugates. SEM images of as formed films containing a. graphene, b. graphene-hemin, c. graphene-GOx and d. graphene-hemin-GOx; and the respective films after immersing into platelet rich rabbit blood plasma for 3 days: e. graphene, f. graphene-hemin, g. graphene-GOx and h. graphene-hemin-GOx. Films containing graphene-hemin-GOx exhibit a minimum morphology change by SEM after immersion into blood plasma compared to control films of graphene, graphene-hemin or graphene-GOx. Scale bars are 10 μm.

FIG. 5 shows FT-IR spectrum of headspace gas from L-arginine oxidation reaction vessel (with graphene-hemin catalysts). Nitrous oxide with two stretching bands at about 2211 cm⁻¹ and about 2235 cm⁻¹ are present. The NO stretching band at about 1790 cm⁻¹ and about 1810 cm⁻¹ are not observed.

FIG. 6 shows GC-MS analysis of headspace gas from L-arginine oxidation reaction vessel (with graphene-hemin catalysts). (a) GC profile of headspace gas. (b) MS profile of headspace gas at retention time of about 3.69 min, indicating the presence of CO₂. (c) MS profile of headspace gas at retention time of about 4.06 min, indicating the presence of nitrous oxide.

FIG. 7 shows chemiluminescence analysis of the oxidation reaction product and the standard NO solution. (a) blank NO solution. (b) NO solution incubated with graphene-hemin conjugates. (c) NO solution incubated with graphene-hemin-GOx conjugates. All three peaks show the same intensity, demonstrating catalyst conjugates don't trap NO to a detectable degree. Chemiluminescence experiments don't show any detectable NO signal from the product of the L-arginine oxidation catalyzed by the conjugates. Control experiments with standard NO solution with or without the catalyst conjugates show similar intensity, demonstrating that the catalyst conjugates also do not trap NO to a detectable degree, which further exclude NO as a possible product.

FIG. 8 shows MS spectrum of L-citrulline detected by LC-MS. The detected L-citrulline shows a protonated molecular ion peak [M+H]⁺ (m/z 176), which has the highest abundance, together with several signature peaks.

FIG. 9 shows stained TEM images. (a) graphene-hemin. (b) GOx. (c,d) graphene-hemin-GOx. Dark features populated on graphene sheets (mostly near the edges) are GOx linked with carboxyl groups around the edge or defect sites of graphene via NHS/EDC coupling. Scale bars are 40 nm.

FIG. 10 shows H₂O₂ evolution profile of graphene-hemin-GOx. The H₂O₂ production rate is about 0.83 μM/min per mg conjugates.

FIG. 11 shows (A) a schematic of a biocompatible film comprising the graphene-catalysts conjugate described herein, and (B) a schematic of a biomedical device comprising the graphene-catalysts conjugate described herein.

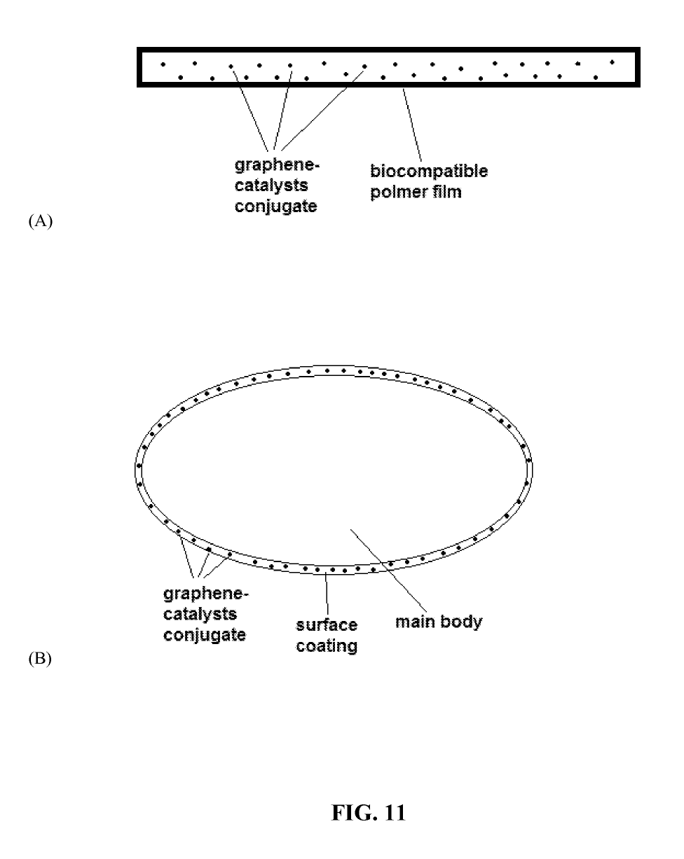

### DETAILED DESCRIPTION

Conjugation of multiple (e.g., two or more) different catalyst systems on a common platform support offers a pathway to drive reactions under physiologically relevant conditions. In this regard, graphene represents an interesting support for both enzymatic and molecular catalysts due to several of its characteristics. As will be understood, graphene is an allotrope of carbon, and its structure is typically one-atom-thick sheets of sp²-bonded carbon atoms that are packed in a honeycomb crystal lattice. In some embodiments, graphene is provided in the form of thin films of a monolayer of carbon atoms that can be envisioned as unrolled carbon nanotubes, although a bilayer or other multilayer of graphene is also encompassed by this disclosure.

First, bulk quantities of graphene flakes can be prepared through chemical exfoliation of graphite oxide (GO) followed by chemical reduction. Chemically reduced graphene typically possesses a large number of functional groups at the edges or defect sites to allow solubility/dispersibility in various solvents. These functional groups can also allow flexible covalent chemistry for linkage with molecular systems or enzymes. Additionally, the extended it surface of graphene can also allow further functionalization via cation-π or π-π interactions. This rich surface chemistry offers excellent potential for coupling multiple different catalysts on graphene to create tandem catalysts for reaction cascading. Furthermore, the two-dimensional structure of graphene provides a desirable geometry as a catalyst support with a large open surface area that is readily accessible to substrates/products with minimal diffusion barriers. Finally, graphene has better biocompatibility than other carbon nanomaterials for potential biomedical applications. In some embodiments, graphene is used as a platform support for both enzymatic and molecular catalysts to create an integrated tandem catalytic system for sustained generation of antithrombotic species.

Thrombus formation is one of the most common and severe problems that lead to complications of blood-contacting biomedical devices including catheters, vascular grafts, biosensors, and heart valves. Therefore, it is of considerable interest to develop an antithrombotic coating on biomedical devices that can sustain their functionality, decrease failure rate, and thereby greatly reduce associated medical complications and cost. Nitric oxide (NO) is a potent antiplatelet agent that can help prevent thrombus formation. The extraordinary thrombo-resistant nature of the inner walls of healthy blood vessels is largely attributed to the continuous production of low fluxes (about 0.5˜4.0×10⁻¹⁰ mol cm⁻² min⁻¹) of NO by endothelial cells (ECs) that line the inner walls of blood vessels. Polymeric coatings capable of releasing or generating NO are of interest for mitigating the risk of thrombus formation. Exogenous NO donors, such as diazeniumdiolates (NONOates), can quickly release NO when exposed to water or physiological environments (e.g., blood, body fluids, and so forth). Such polymeric coatings with embedded or covalently linked NO donors release NO to minimize thrombus formation. However, the application of this approach for long-term implants, such as vascular grafts or hemodialysis catheters, is constrained by the inevitable depletion of the finite reservoir of reagents in an exogenous NO donor source. In addition, the labile nature of many NO donors (heat, light, and moisture sensitivity) curtails their practical manufacturability and clinical applications. Moreover, the toxicity of some diazeniumdiolate precursors and the potential formation of carcinogenic nitrosamine byproducts may also pose an adverse effect. Alternatively, a surface coating capable of catalytic generation of NO from physiological components may offer a more attractive strategy for sustained NO release. For example, organoselenium can trigger the decomposition of S-nitrosothiols (RSNO), which are endogenous NO carriers, to generate NO; this strategy is potentially useful for the continuous release of NO over long time periods. However, the relatively low level and highly variable concentrations of endogenous RSNOs in blood can constrain the reliability of these NO generating materials. In vivo toxicity studies also indicate that the reaction between reduced selenium species and oxygen is fast enough to produce a significant amount of superoxide that can react with NO to produce peroxynitrite, a toxic species. In addition, selenium radical formation is also problematic, although aromatic organoselenium species have been found to be far less toxic (e.g., ebselen).

Biologically, NO is believed to arise from the oxidation of L-arginine catalyzed by a family of nitric oxide synthase (NOS) enzymes that utilize the reduced form of nicotinamide adenine dinucleotide phosphate (NADPH) as a cofactor along with O₂ as the oxidant. Under conditions where either, or both, the L-arginine concentration and cofactor supplies are limited, nitroxyl (HNO), the one electron reduced form of NO, can also be produced. Nitroxyl possesses antithrombotic activity similar to NO, and hydrogen peroxide can substitute for NADPH and O₂ as the oxidant for nitroxyl production. Thus biomimetic generation of antithrombotic nitroxyl is a solution to the problems associated with NO releasing materials and NO generating catalysts. Therefore, a biocompatible surface capable of local generation of nitroxyl for effectively minimizing thrombus formation is desirable.

In some embodiments, the immobilization of hemin together with glucose oxidase enzyme (GOx) onto solid graphene supports is used to create an integrated tandem catalytic system that can make use of endogenous materials in blood for the sustained biomimetic generation of nitroxyl. Hemin, an iron porphyrin species, is the catalytic center of NOS. Free hemin itself is generally inactive as a catalyst because it undergoes molecular aggregation and oxidative destruction under physiological conditions. Resin-supported hydrophilic iron porphyrin derivatives can be active for the oxidation of L-arginine, but with a rather limited turn-over number due to a rapid loss of catalytic activity. This system is also not suitable for practical clinical applications because it involves a high concentration of H₂O₂ oxidant (e.g., 38 mM), well beyond the physiological concentration. Monomeric hemin can be immobilized onto graphene to form a stable graphene-hemin conjugate that exhibits peroxidase-like activity for a variety of biomimetic oxidation reactions, using H₂O₂ as the oxidant. In the tandem catalyst system of some embodiments, with the integration of GOx, H₂O₂ is produced locally from endogenous glucose for the subsequent hemin-catalyzed oxidation of L-arginine to generate antithrombotic nitroxyl species.

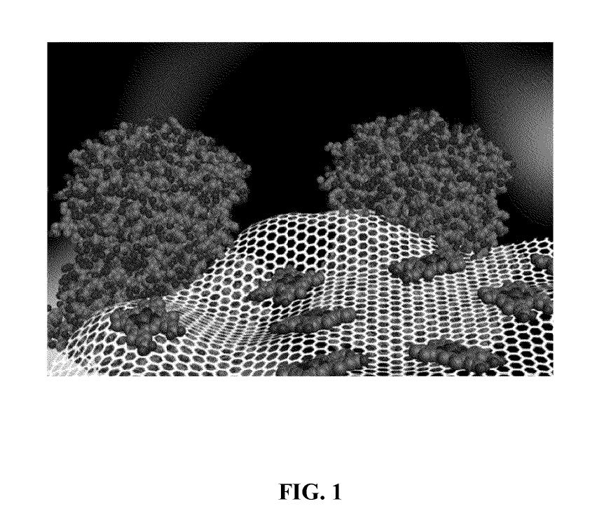

In some embodiments, monomeric hemin is immobilized onto graphene through π-π interactions, and GOx is covalently linked with graphene to form a graphene-hemin-GOx conjugate surface (FIG. 1). Furthermore, it is demonstrated that this complex conjugate can be used as an effective biomimetic catalyst for the generation of nitroxyl species using only or primarily endogenous species, namely glucose and L-arginine. Of note, the physiological concentrations of glucose and L-arginine and the nitroxyl levels for antiplatelet activity follow a nearly ideal cascade: blood glucose concentration is about 2-5 mM, capable of creating more than enough peroxide to oxidize L-arginine, which is present at about 200 μM. The amount of nitroxyl for significant antithrombotic effects is likely in the sub-μM range, which is at least three to four orders of magnitude lower than actual L-arginine concentrations. The nitroxyl production behavior is investigated using various methods including Fourier transform-Infrared (FT-IR) spectroscopy, Gas chromatography-Mass spectrometry (GC-MS), fluorescence spectroscopy (diaminofluorescein (DAF) assay), and chemiluminescence. Furthermore, a biocompatible polymer film with embedded graphene-hemin-GOx conjugates was prepared, and demonstrated to exhibit greatly enhanced anti-platelet activity due to the continuous generation of nitroxyl.

Nitroxyl Production by Graphene-Hemin Conjugates.

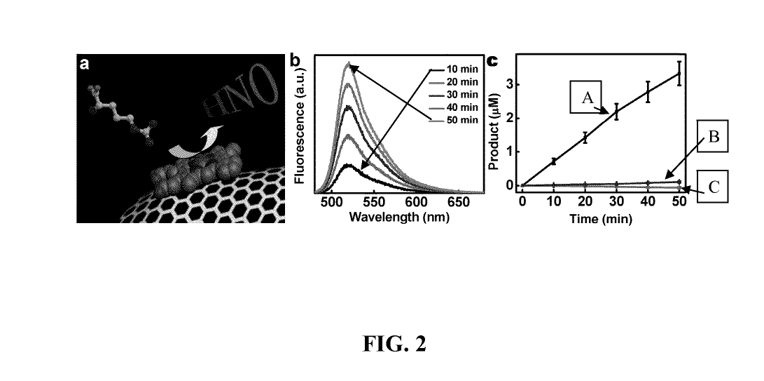

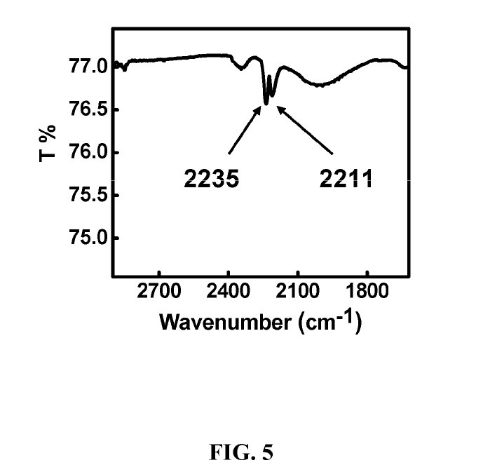

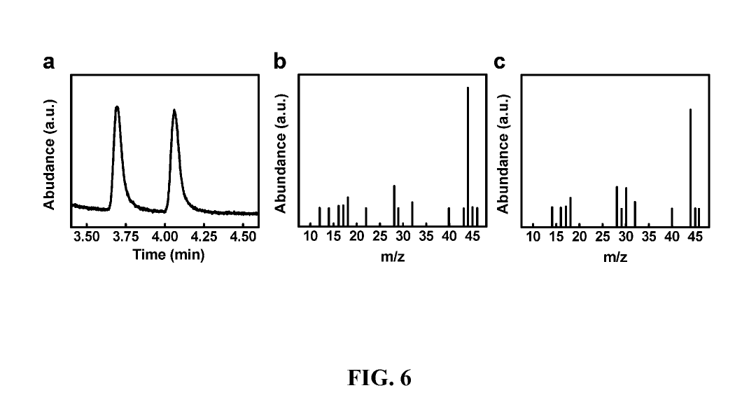

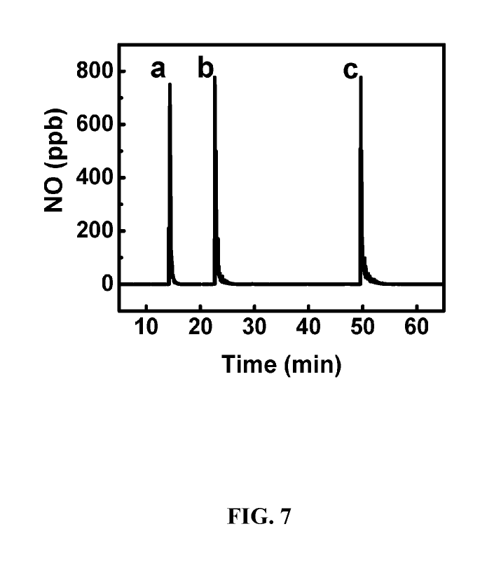

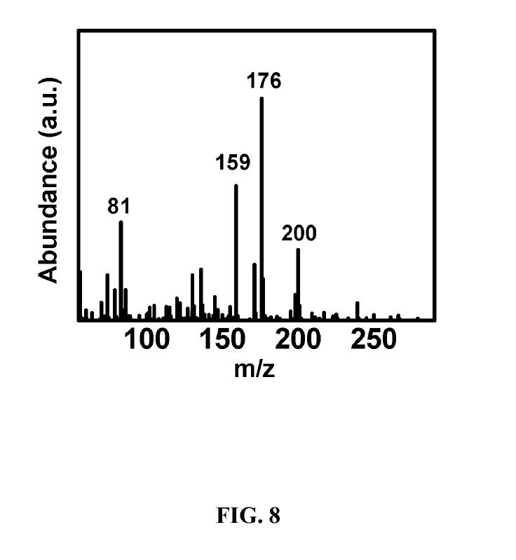

The catalytic oxidation characteristics of graphene-hemin conjugates were initially investigated. Graphene was obtained by hydrazine reduction of exfoliated graphene oxide prepared via Hummer's method. The immobilization of monomeric hemin on graphene via π-π stacking was conducted using the approach set forth in Xue, T. et al., “Graphene-supported hemin as a highly active biomimetic oxidation catalyst,” Angewandte Chemie-International Edition 51, 3822-3825, (2012), the disclosure of which is incorporated herein by reference in its entirety. L-arginine oxidation reactions were conducted by dispersing the graphene-hemin catalyst in a pH 7.4 Phosphate buffered saline (PBS) buffer with about 200 μM L-arginine added, along with about 5 mM H₂O₂ as the oxidant (FIG. 2a). The L-arginine oxidation reaction could potentially result in multiple different products including nitric oxide (NO) or nitroxyl (HNO). Extensive characterizations demonstrate that the product is predominantly HNO. For product identification, the generated nitroxyl dimerizes to form nitrous oxide over time, which is detectable by gas phase FT-IR spectroscopy. The gas phase FT-IR spectrum of the headspace gas of a reaction vessel confirms the presence of nitrous oxide with two stretching bands at about 2211 cm⁻¹ and about 2235 cm⁻¹ (see FIG. 5). The NO stretching bands at about 1790 cm⁻¹ and about 1810 cm⁻¹ are not observed, excluding NO as the product of the reaction. The headspace gas is also tested by GC-MS for nitrous oxide detection, which further establishes the existence of nitroxyl (see FIG. 6). Additionally, chemiluminescence analysis, which can selectively detect parts-per-billion (ppb) levels of NO, but not nitroxyl nor nitrous oxide, also demonstrates that no detectable NO is produced from the oxidation reaction (see FIG. 7). The expected byproduct L-citrulline is also tested by Liquid chromatography-Mass spectrometry (LC-MS), further confirming the reaction pathway (see FIG. 8).

The above studies demonstrate that a graphene-hemin conjugate can function as an effective catalyst for the production of nitroxyl. To quantify the generated nitroxyl amount in reaction solution, a fluorescence DAF assay was utilized. Nitroxyl can react with DAF-2 to form DAF-triazole with fluorescence emission. The fluorescence spectrum was monitored at different time intervals (FIG. 2b), and the intensity increase of the emission peak at about 515 nm was calibrated with the corresponding nitroxyl concentrations (FIG. 2c). The DAF assay shows the production of nitroxyl immediately after the introduction of H₂O₂ to a graphene-hemin catalyzed reaction mixture, while the control reaction without the graphene-hemin conjugate does not yield any detectable signal (FIG. 2c). Of note, for the reactions with the equivalent amount of hemin, the graphene-hemin catalysts exhibit a remarkably higher activity, while the free hemin hardly shows any catalytic activity (FIG. 2c). Such a difference in catalytic behavior can likely be attributed to the monomeric molecular structure of hemin on graphene supports. For free hemin, the active catalytic sites are limited due to molecular aggregation of hemin to form inactive dimers. The catalytic turn-over frequency of graphene-hemin is calculated to be about 0.015 min⁻¹ (FIG. 2c), which is greatly higher than that of a resin supported system (0.0016 min⁻¹). Moreover, graphene-hemin conjugates also exhibit exceptional catalytic activity stability, with nearly a constant turnover rate over a 50-minute test period, while the resin supported hemin can catalyze the reaction for about 6 min before a total loss of its catalytic activity.

Synthesis of Graphene-Hemin-GOx and Nitroxyl Production from L-Arginine and Glucose.

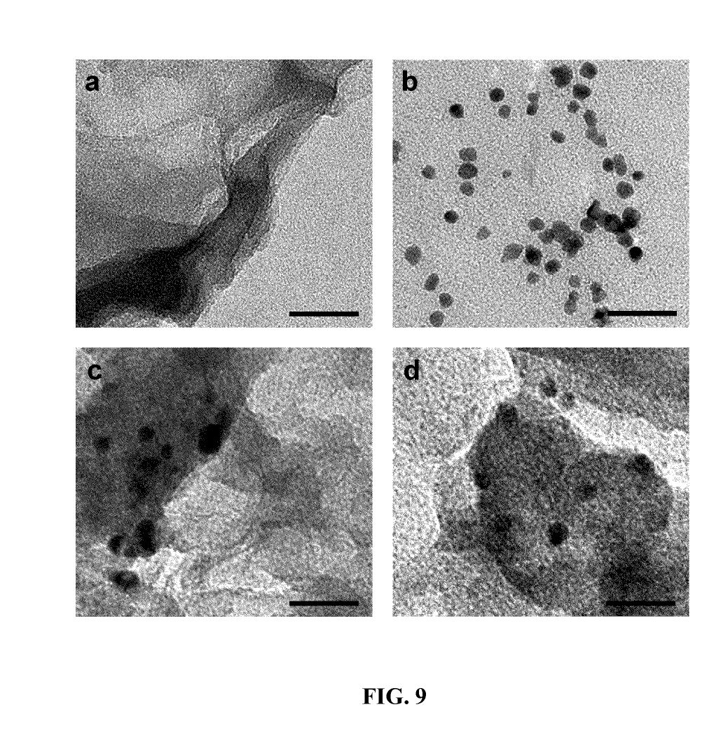

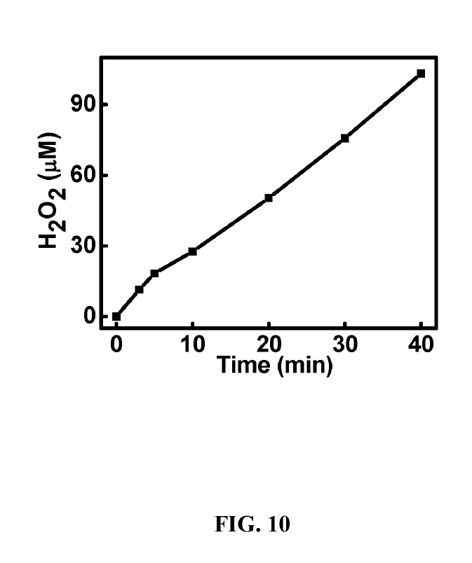

Although, as demonstrated above, the graphene-hemin conjugates can effectively catalyze the oxidation of L-arginine to generate nitroxyl, this reaction involves a relatively high concentration (about 5 mM) of H₂O₂ oxidant that is far above the physiological H₂O₂ concentration (about 10⁻⁹ to about 10⁻⁷ M). To apply the graphene-hemin catalyst for practical applications under physiological conditions, a mechanism to locally produce desired levels of H₂O₂ is desired. To this end, linking GOx to the graphene-hemin conjugates can offer an approach to elevate the local H₂O₂ concentration through the oxidation of blood glucose. GOx was anchored via a N-Hydroxysuccinimide and 1-Ethyl-3-(3-dimethylaminopropyl)carbodiimide (NHS-EDC) coupling reaction and linked to the edge and defect site carboxyl groups of graphene. Stained TEM shows dark features of about 10 nm size distributed around the edges or defective sites of graphene, which is attributed to the successful linkage of GOx (see FIG. 9). The formation of graphene-hemin-GOx is also supported by zeta potential measurements (see Table 1 below). Once the graphene-hemin-GOx conjugates were obtained, H₂O₂ production activity was tested in the presence of glucose and L-arginine (see FIG. 10), demonstrating that GOx retains good activity after chemical linkage onto the graphene.

TABLE 1
Zeta potentials are measured at 25° C. in pure water. Graphene-
hemin has a more negative potential than graphene, due to the conjugation
of negatively charged hemin. Graphene-hemin-GOx is slightly more
negative than graphene-hemin, indicating the successful
conjugation of GOx.

| Materials | Zeta potential (mV) |
|---|---|
| GOx | −4.14 |
| graphene | −24.2 |
| graphene-hemin | −28.5 |
| graphene-hemin-GOx | −31.6 |

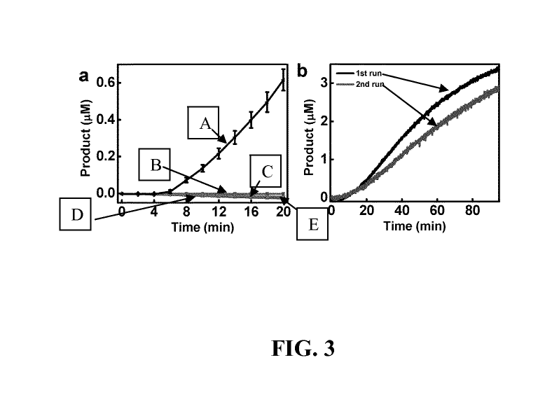

The integrated catalysts were then used to catalyze the L-arginine oxidation reaction, and the nitroxyl generation behavior was studied using the DAF-based nitroxyl assay. In pH 7.4 PBS buffer containing physiological concentrations of glucose (about 5 mM) and L-arginine (about 200 μM), the graphene-hemin-GOx conjugates produce nitroxyl after a short activation stage (about 5 min) (FIG. 3a). This lag might be due to the accumulation of an adequate H₂O₂ concentration at the surface of the graphene. For a series of control experiments, graphene-hemin-GOx catalysts in a solution of only glucose cannot produce any nitroxyl, and a similar result was obtained for a solution containing only L-arginine, but not glucose (FIG. 3a). In a solution with both glucose and L-arginine, if graphene-hemin conjugates or graphene-GOx conjugates alone are introduced, no nitroxyl production is observed. Taken together, these findings demonstrate that nitroxyl production is observed when the graphene-hemin-GOx conjugates, glucose, and L-arginine are all present (FIG. 3a). The real time reaction behavior of this mixture was also monitored (FIG. 3b). Overall, the graphene-hemin-GOx conjugates can maintain good and stable activity over an extended period, and exhibits excellent recyclability (FIG. 3b).

Antithrombotic Behavior of Graphene-Hemin-GOx in Contact with Blood Plasma.

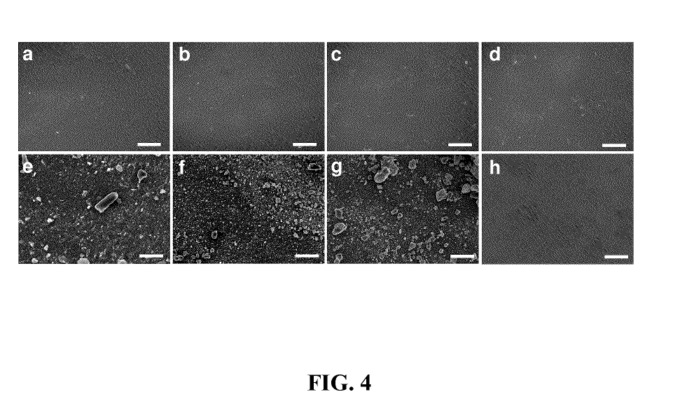

The studies have demonstrated that graphene-hemin-GOx conjugates can function as effective catalysts for the generation of nitroxyl with endogenous components. To further investigate whether the graphene-hemin-GOx conjugates can offer an effective solution for biomedical applications, the conjugates are embedded (at about 40 wt %) in a commercially available polyurethane (available under the brand Tecophilic® SP-93A-100) that was then spin-coated to form biocompatible films. Control thin film samples were also prepared with embedded graphene, graphene-hemin or graphene-GOx (at the same wt %). All the films were then immersed into platelet rich rabbit blood plasma for 3 days, and then examined by scanning electron microscopy (SEM) to evaluate the platelet adhesion characteristics. Control films containing graphene, graphene-hemin or graphene-GOx exhibited very rough surfaces after blood contact, indicating adhesion of a significant number of blood platelets (FIG. 4a-c, e-g). In contrast, the film containing graphene-hemin-GOx shows a minimum morphology change before and after blood contact (FIG. 4d,h), demonstrating excellent anti-platelet function.

By simultaneously conjugating hemin and glucose oxidase on graphene, an integrated tandem catalyst is provided that can drive a reaction cascade to allow for in-situ generation of H₂O₂ for the oxidation of L-arginine. This process can thus allow sustained generation of nitroxyl from physiological glucose, L-arginine and blood oxygen. Embedding of such tandem catalysts into biocompatible films can create a surface coating with excellent anti-platelet characteristics, offering a solution to sustained generation of antithrombotic nitroxyl species on medical devices when in contact with fresh blood. Overall, the studies demonstrate a general strategy to integrate molecular catalysts and enzymatic catalysts on the same platform for them to synergistically facilitate complex reaction pathways under mild physiological relevant conditions, and allow important chemical transformations not otherwise readily possible. It can impact diverse areas including biomedicine and green chemistry.

More generally, a variety of combinations of different catalysts can be conjugated on graphene, including a combination of at least one molecular catalyst and at least one enzymatic catalyst, a combination of two or more different molecular catalysts, and a combination of two or more different enzymatic catalysts. In some embodiments, enzymatic catalysts correspond to, or include, proteins or other sequences of amino acid residues. In some embodiments, molecular catalysts correspond to, or include, a non-protein or non-peptide chemical compound, wherein optionally the molecular catalysts are substantially or totally free of amino acid residues. In some embodiments, a molecular catalyst has a molecular weight of 1,500 g/mol of less, while an enzymatic catalyst has a molecular weight greater than 1,500 g/mol. In addition to, or in place of, hemin, other organometallic catalysts, including other metalloporphyrins or any molecular catalysts which can stack onto the surface of graphene, can be used. In addition to, or in place of, glucose oxidase, another oxido-reductase that catalyzes the oxidation of glucose to hydrogen peroxide or any other molecular or enzymatic catalysts which can covalently link onto graphene edges and defects can be used. The conjugation of catalysts onto graphene can be attained through a variety of types of bonding, including π-π interactions, covalent linkages, cation-π interactions, and combinations thereof. In addition to, or in place of, polyurethane, tandem catalysts can be embedded into biocompatible films of a variety of biocompatible polymers or other materials for use in biomedical devices such as catheters, vascular grafts, biosensors, and heart valves. A biocompatible film can be implemented as a surface coating on a main body portion of a biomedical device, or can be implemented as a part of the main body portion. The tandem catalysts can also be used in wide areas where cascading reactions are desired, such as photochemistry, energy harvesting, and so forth. Moreover, graphene also has the ability to tune the electron density of the conjugated molecular/enzymatic catalysts which could further enhance the catalytic behavior. Lastly, graphene can serve as an electron channel to transfer electrons from one catalyst to another, which can be used as an ideal support for the design of tandem catalyst systems involving electron transfer, such as multi-step photochemistry.

### Working Examples

### Preparation of Graphene-Hemin-GOx Conjugates

The preparation of graphene-hemin-GOx conjugates are followed stepwise via immobilization of hemin on graphene surface, then linkage of GOx to the carboxyl groups at edge and defect site of graphene. The graphene-hemin conjugates were prepared using the approach set forth in Xue, T. et al., “Graphene-supported hemin as a highly active biomimetic oxidation catalyst,” Angewandte Chemie-International Edition 51, 3822-3825, (2012), which was referenced above. Graphene-hemin conjugates were then mixed with NHS-EDC coupling agent for about 2 hours, centrifuged and washed, followed by stirring with about 0.1 mg/mL GOx in pH 7.4 PBS buffer overnight for GOx linkage. Graphene-hemin-GOx conjugates were then centrifuged and washed with a pH 7.4 PBS buffer.

Characterization of L-Arginine Oxidation Reaction.

L-arginine oxidation reactions by graphene-hemin were carried out in the presence of about 200 μM L-arginine and about 5 mM H₂O₂ in a pH 7.4 PBS buffer. L-arginine oxidation reactions by graphene-hemin-GOx were carried out in the presence of about 200 μM L-arginine and about 5 mM glucose in a pH 7.4 PBS buffer. The product was characterized using FT-IR, GC-MS, DAF assay, and chemiluminescence. For FT-IR spectroscopy, the gas phase FT-IR spectrum of the headspace gas was taken after 2-hour reaction. For GC-MS measurement, the headspace gas was injected into an Agilent 6890-5975 GC-MS with a 30 m Rt®-Q-Bound column (Resteck Co, Columbia, Md.) at an operating oven temperature of about 45° C. under about 14.6 psi He carrier gas. For the DAF Assay, about 10 μM DAF-2 was added to the reaction solution. The excitation wavelength was about 448 nm. At each time interval, florescence spectra were obtained from an average of five accumulations. Peak intensities of about 515 nm were also monitored continuously for reaction catalyzed by graphene-hemin-GOx conjugates. For chemiluminescence, the solution after 2-hour reaction was bubbled with argon, and the products were measured via a chemiluminescence NO Analyzer™, Model 280 (Sievers Instruments, Boulder, Colo.). In situ measurements were also carried out.

Antithrombotic Film Fabrication and Antithrombotic Studies.

Tecophilic® SP-93A-100 polyurethane was dissolved in tetrahydrofuran (THF) to make a solution of about 40 mg/mL. Graphene, graphene-hemin, graphene-GOx or graphene-hemin-GOx were then mixed with the polymer solution, and films were cast on silicon substrates by spin-coating. Films were peeled off after drying. Arterial blood from New Zealand white rabbits, weighing 2.5-3 kg, was drawn into a 9:1 volume of a blood:anticoagulant citrate solution. NIH guidelines for the care and use of laboratory animals (NIH Publication no. 85-23 Rev. 1985) were observed throughout. The citrated whole blood was centrifuged at about 110 g for about 15 min at about 22° C. Platelet-rich plasma was collected from the supernatant. Films were first immersed in a pH 7.4 PBS buffer containing about 200 μM L-arginine and about 5 mM glucose for about 30 min, then immersed in platelet-rich plasma for 3 days. Films were then washed with pH 7.4 PBS buffer, dried and sputtered with gold for platelet aggregation and thrombus formation investigation by JEOL JSM-6700F FE-SEM.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

1. A graphene-catalysts conjugate, comprising:
a graphene support;
a first catalyst conjugated to the graphene support; and
a second catalyst conjugated to the graphene support, and the second catalyst is different from the first catalyst.

2. The graphene-catalysts conjugate of claim 1, wherein the first catalyst and the second catalyst correspond to a tandem catalytic system to drive a chemical transformation.

3. The graphene-catalysts conjugate of claim 1, wherein the first catalyst is a molecular catalyst.

4. The graphene-catalysts conjugate of claim 1, wherein the first catalyst is an organometallic catalyst.

5. The graphene-catalysts conjugate of claim 1, wherein the first catalyst is a metalloporphyrin.

6. The graphene-catalysts conjugate of claim 1, wherein the first catalyst is hemin.

7. The graphene-catalysts conjugate of claim 1, wherein the first catalyst is conjugated to the graphene support through π-π interactions.

8. The graphene-catalysts conjugate of claim 1, wherein the second catalyst is an enzymatic catalyst.

9. The graphene-catalysts conjugate of claim 1, wherein the second catalyst is an oxido-reductase.

10. The graphene-catalysts conjugate of claim 1, wherein the second catalyst is glucose oxidase.

11. A biocompatible film, comprising:
a polymer film; and
a graphene-catalysts conjugate of claim 1 embedded in the polymer film.

12. A biomedical device, comprising:
a main body portion; and
a surface coating on the main body portion, and the surface coating includes a graphene-catalysts conjugate of claim 1 embedded in the surface coating.

13. The biomedical device of claim 12, which is an implant.

14. The biomedical device of claim 12, which is a catheter, a vascular graft, a biosensor, or a heart valve.

15. A method for making the graphene-catalysts conjugate of claim 1, comprising covalently linking the second catalyst to a conjugate of the graphene support and the first catalyst.

16. The method of claim 15, wherein the first catalyst is hemin which is conjugated to the graphene support through π-π interactions.

17. The method of claim 16, wherein the second catalyst is glucose oxidase, and wherein the glucose oxidase is covalently linked to the graphene support through a coupling agent.

18. A method for making the biocompatible film of claim 11, comprising coating a surface with a composition comprising the polymer mixed with the graphene-catalysts conjugate.

19. A method for making the biomedical device of claim 12, comprising coating the main body portion with a composition comprising the graphene-catalysts conjugate.

20. A method for improving the antithrombotic property of an implant, comprising coating the implant with a composition comprising the graphene-catalysts conjugate of claim 1.
